(19) United States
(12) Patent Application Publication
(10) Pub. No.: US 2002/0090733 A1
(43) Pub. Date: Jul. 11, 2002

(54) Process for preparing flavour compounds

(75) Inventors: Werner Klaffke, Vlaardingen (NL); Shail Patel, Bebington (GB); Jeremy Andrew Rabone, Bebington (GB); Stephen William Russell, Vlaardingen (NL); Johannes Theodorus Tissen, Vlaardingen (NL)

Correspondence Address: UNILEVER, PATENT DEPARTMENT, 45 RIVER ROAD, EDGEWATER, NJ, 07020, US

(73) Assignee: Lipton, Division of Conopco, Inc.

(21) Appl. No.: 09/909,635

(22) Filed: Jul. 20, 2001

(30) Foreign Application Priority Data
Jul. 21, 2000 (EP) 00306249.4

(51) Int. Cl. G05B 21/00; G01N 33/00

(52) U.S. Cl. 436/89; 700/266

(57) ABSTRACT

A process for preparing components or mixtures of components that can be utilised to impart a flavour to a foodstuff. More in particular, a process for preparing flavour components involving so-called Maillard reactions, wherein at least part of the reaction conditions is determined by modelled relationships and/or a simulation of complex chemical reaction pathways.

### FIELD OF THE INVENTION

The present invention relates to a process for preparing components or mixtures of components that can be utilised to impart a flavour (i.e. taste and/or aroma) to a foodstuff. More in particular, this invention relates to a process for preparing flavour components involving so-called Maillard reactions, wherein at least part of the reaction conditions and/or reactants is determined by modelled relationships and/or a simulation of complex chemical reaction pathways.

### BACKGROUND OF THE INVENTION

When carbohydrates (sugars or source thereof, like e.g. polysaccharides or unspecified glycosides) and amino acids (or source thereof, such as protein, glycoprotein, peptide), or materials containing sugars and amino acids are heated under specific conditions (pH, t, T, etcetera), a “soup” containing a wide variety of reaction products can be formed. Many of the formed reaction products, in particular the more volatile components, are known to have properties that make them desirable in flavour and/or aroma compositions, e.g. for use in foods and drinks. Such compounds are known as Maillard flavours.

Maillard flavours can be savoury flavours, although many can also be described as having a nutty, caramel, jammy, burnt, sulphury, or other character. Maillard flavours are formed in normal cooking processes, such as e.g. frying (deep or shallow), roasting and grilling. Examples are the frying of meat, or the formation of an “au gratin” crust of heated cheese.

Flavour and/or aroma compositions containing Maillard flavour and/or aroma components are used in many fields of food processing. Almost all flavour suppliers have a range of Maillard-type flavour compositions for flavouring purposes. Such compositions are usually prepared by heating minimally a sugar (or carbohydrate source) together with a nucleophilic species, such as biogenic amines, amino acid(s), or sources of amino acids such as peptides or proteins and their hydrolysates or extracts, e.g. HVP, yeast extracts and hydrolysates, or soy sauces, at certain conditions. This may further contain additives such as salts, cosolvents, buffers, other compounds which are found on the FEMA GRAS list such as aldehydes, ketones, alcohols, amines, organic acids, esters and lactones, explicitly fatty acids or esters thereof, cyclic heterocycles, thiols, ethers, thioethers such that Maillard flavours are formed (together with non-volatile compounds of usually higher molecular weight or polarity, as well as insoluble coloured polymeric material). Also may be present: HVP, fats, yeast extracts, meat extracts, other hydrolysates, soy sauce, peptones, fatty acids and fractions thereof. Said flavouring compounds may also be prepared starting from reaction products of carbonyl compounds (as defined above) and a nucleophilic species (as defined above). For the purpose of this invention, the above is summarised as “reactants”. Whenever “reactants” is used throughout this patent description, the description of the group of compounds above is encompassed.

Numerous reactions take place during the heating process aimed at production of Maillard flavours, due to the fact that amino acids and sugars can react in different ways, but also because the reaction products in turn can undergo further reaction (chain reaction), and also because many different sugars and different amino acids give different reaction products, having different sensorical properties. Also, processing conditions such as the duration of the reaction (t), the temperature and temperature profile (T(t)), the acidity and water activity of the reaction mixture (pH, a_{w}), and others have a considerable influence on the resulting Maillard flavour and its character.

Although Maillard flavours and their formation have been the subject of many studies, the preparation of Maillard flavour compositions is still very much an empirical job, involving trial and error, blended with experience. If a specific Maillard flavour character is desired by a food manufacturer it is not at all clear what the composition of the starting materials must be, let alone the processing conditions, to arrive at the desired end result. A higher degree of predictability is desired, and if possible guidance as to what the conditions and starting material and their concentrations should be to arrive at a desired end result.

### SUMMARY OF THE INVENTION

Hence, there was a need for a process for preparing Maillard-type flavour compounds, in which at least a number of the processing parameters (such as the relative concentrations of the reactants, t, T, pH, etcetera) and also the reactants themselves are to at least some extent derivable from a relationship with the desired end result. In other words, there is a need for a process for preparing Maillard-type flavour components in which, if the character of the desired result is described in sensorical properties, there is some guidance as to what the starting materials and reaction conditions need to be to arrive at or near the desired end result.

It has now been found that the above objective can to a considerable extent be achieved by a process for preparing a volatile component or mixture of components, said process comprising heating a mixture containing reactants as herein defined, said heating being performed under N different processing parameters wherein one or more of the N processing parameters and the reactant(s) to choose for obtaining the desired volatile component(s) are derivable from a relationship between:

sensorical data obtainable from analysing reaction products of actual experiments,

volatile reaction products and their composition,

processing parameters,

reactants,

said composition being obtainable:

(a) from an actual mass distribution obtainable from performing at least 100 (preferably at least 1000) reactions involving heating reactants as herein defined under predetermined and known processing parameters, analysing the volatile fraction of the reaction product obtained from each of the reactions above to provide composition analyses thereof, encoding it as a mass distribution, and/or

(b) by simulation of complex chemical reaction pathways involving the simulation of reacting reactants as herein defined under predetermined and specified processing parameters.

Said N different processing variables may comprise at least one of concentration of reactants, heating temperature, heating time, pH of the reaction mixture, water activity and optionally further added salts, cosolvents, and buffers. Next to the N different processing variables are the variables of the reactant(s) (i.e. which type and structure).

The reactants are herein to be understood to comprise minimally a sugar (or carbohydrate source from natural material or hydrolysates thereof) together with a nucleophilic species, such as biogenic amines, amino acid(s), or sources of amino acids such as peptides or proteins and their hydrolysates or extracts, e.g. HVP, yeast extracts and hydrolysates, or soy sauces, at certain conditions. This may further contain additives such as salts, cosolvents, buffers, other compounds which are found on the FEMA GRAS list such as aldehydes, ketones, alcohols, amines, organic acids, esters and lactones, explicitly fatty acids or esters thereof, heterocycles, thiols, ethers, thioethers such that Maillard flavours are formed (together with non-volatile compounds of usually higher molecular weight or polarity, as well as insoluble coloured polymeric material). Said flavouring compounds may also be prepared starting from reaction products of carbonyl compounds (as defined above) and a nucleophilic species (as defined above).

Optionally, the mass distribution in (a) and (b) may be checked for isomeric structures by comparing experimentally obtained mass spectra and the molecular description (e.g. SMILES) as a result from the simulation.

### DETAILED DESCRIPTION OF THE INVENTION

There are a number of approaches in the literature which simulate reaction pathways either synthetically or retro-synthetically. These may be summarised as:

(i) Search engines based on large databases, e.g. CASREACT, CRDS, BEILSTEIN, ORAC, REACCS, SYNLIB, and CHEMINFORM which classify reactions and allow searches by molecule fragments and functional groups.

(ii) Computer-aided Synthesis, e.g. PSYCHO, DARC-SYNOPSIS and REACTION simulates reactions in the forward direction from start reactants.

(iii) Computer-aided Retro-synthesis e.g. LHASA, RETROSYN, OCSS and SYNCHEM, builds the synthetic tree for a user-specified molecule. Some also support synthesis in the forward direction, i.e. allow the user to specify start compounds to predict end products e.g. SOS^{[4]}, MARS^{[5]} and SYNGEN.

(iv) Mathematical models, e.g. energy calculations (EROS) or electron density calculations (CAMEO), are used to predict chemical reactions.

(v) Combinatorial Chemistry e.g., Diversity Explorer^{[1]}, Chem-X^{[2]}, or Legion,^{[3]}, for building virtual combinatorial libraries.

Bador^{[6]} et al. give a review of the approaches listed under (i) to (iv).

As the intended use of these approaches is generally an aid for the synthetic chemist, they have common drawbacks such as: user input is required to proceed, and/or only a single branch of the reaction pathways is followed, or other disadvantages.

In order to predict the outcome of processes that involve multiple chain reactions, a system that can cope with inherent parallelism and feedback loops, and operate without user interaction to construct the complete reaction graph, is preferred. Prickett and Mavrovouniotis^{[7]} have developed a theoretical system that models generic complex reaction systems. This iteratively applies known elemental reaction steps, according to theoretical chemistry, to the reactants and all intermediates.

The system according to the present invention is similar to this, but better in three ways:

1) taking into account reaction rate constants as reaction probabilities

2) and optionally heuristic blocking of the reactions into subsets that guide the reactions in a computationally effective manner

3) and optionally fine tuning the reaction and rate databases by comparison with experimental results.

The simulation of complex chemical reaction pathways according to this invention (hereafter called Iterated Reaction Graphs—IRG) models complex reaction pathways by simulating the reaction steps in parallel. An Iterated Reaction Graph has two main data elements:

1. A ‘Soup’ of molecules representing the current state of the system

2. A ‘Reaction Set’ describing transformations (=simulated reactions) that may take place in the Maillard process, and probabilities (=simulated reaction rates of said reactions) to produce volatile and non-volatile molecules.

ad 1) In the ‘Soup’, molecules may be represented by any computer readable format, e.g. expressed as SMILES^{[8]}, a simple line notation of 2-dimensional connection tables. Preferably, during the iterative procedure the newly formed compounds are added back to the Soup, and the volatile components which are present at the simulation form (part of) the virtual mass distribution. Additionally, it is preferred that the Soup at the start of the simulation is equal to the starting mixture of molecules.

ad 2) In order to describe the reactions that may take place in the Maillard process the ‘Reaction Set’ may suitably contain (in computer readable format):

a reaction database, which contains various transformations that may take place under conditions for Maillard-type reactions. These transformations can be found in literature in various review articles^{[13-18]} and the references cited therein.

a reaction kinetic database, containing probabilities for transformations to take place in the reaction database, simulating kinetic data such as rate constants for the reactions.

Furthermore, the IRG contains a computer programme directly loadable in the internal memory of a computer, comprising instructions for the simulation of complex chemical reaction pathways by iteratively applying a set of operations or computer instructions to:

A ‘Soup’ of molecules representing the current state of the system

A ‘Reaction Set’ describing transformations and probabilities that may take place in the Maillard process

to produce volatile and non-volatile molecules, for simulating complex chemical reactions when such product is run on a computer, and wherein the computer programme contains two main elements:

a) computer instructions for applying the transformations using the reaction set described above,

b) computer instructions for the iterative procedure of selecting molecules, applying the transformations and producing output.

The computer programme also contains typical components such as a user interface, methods of inputting and editing data, methods of probing the progress, methods for outputting results and so on.

The invention further comprises a computer program product directly loadable into the internal memory of a digital computer, comprising software code portions for the simulation of complex chemical reaction pathways by iteratively applying a set of operations or computer intructions to:

A ‘Soup’ of molecules representing the current state of the system,

A ‘Reaction Set’ describing transformations that may take place in the Maillard process, with their respective probabilities,

to produce volatile and non-volatile molecules, and wherein the iterative procedure is coded as a computer programme directly loadable in the internal memory of a computer, wherein the iteration is coded as a computer programme, for simulating complex chemical reactions when such product is run on a computer.

Each reaction may be coded as a computer program that takes connection table input (reactants), carries out necessary rearrangements (reactions), and produces a connection table output (products). In the present document such coded (or virtual) reaction is called ‘transformation’.

At a simplistic level the reaction base operates on the molecular soup to form products:

Reaction Set: Molecular Soup→Products

The full complexity of the possible reactions may be modelled by iterating through this ‘equation’, feeding the products back into the Molecular Soup and running through the Reaction Set again, which is a part of the IRG (FIG. 1).

The full reaction graph^{[8-12]}, where molecules are nodes and reactions are arcs may be defined as the set of triplets:

{<Substrate> <Reaction> <Product>}

For example the text below is a small fragment of a Reaction Graph, containing 3 triplets (molecules coded in SMILES):

C(═O)C(C(═CC(═C)O)O)O R1_{—}1_{—}6_endiol C(═C(C(═CC(═C)O)O)O)O

C(═O)C(C(C(C(═C)O)O)O)O R1_{—}1_{—}6_endiol C(═C(C(C(C(═C)O)O)O)O)O

C1═CN═C(C(C)O)O1 R1_{—}4_{—}2_strecker C(═CN)OC(═O)C(C)O

The full graph is reconstructed by linking products to substrates and chaining through the triplets. Examples of two relatively short but different routes to dimethyl pyrazine are given below:

<Start> C(O)C(O)C(O)C(O)C(O)C═O R1_{—}12_{—}3_sugar C(O)C(O)C(O)C(═O)C(═O)C

R1_{—}2_{—}1_retroaldol C(O)C(═O)C(═O)C R1_{—}2_{—}2_retroaldol C═O R2_{—}5_{—}4a_pyrazine

CC-1 NC(C)—CNC-1

<Start> NC(C(O)C)C(═O)O R2_{—}4_{—}1_strecker CC(C═O)N R2_{—}5_{—}11_pyrazine

CC1═NC(C)C═NC1 R1_{—}5_{—}3_pyrazine_oxidation CC-1 NC(C)—CNC-1

The size of the soup, typically 100-1000 molecules, is determined at the start, and is limited only by computer memory considerations. At the start of a run this will be composed of amino acids and sugars only, e.g. for glucose and threonine (coded in SMILES):

“C(O)C(O)C(O)C(O)C(O)C═O

C(O)C(O)C(O)C(O)C(O)C═O

. . .

NC(C(C)O)C(═O)O

NC(C(C)O)C(═O)O

. . . ”

There are duplicates of molecules, as the relative number of times a molecule appears simulates the concentration of that molecule in the soup. During, and at the end of a run, the soup will contain a list of end products that is the result of simulating the reactions many thousands of times. It also may contain duplicates, to simulate the relative concentration of end products, e.g.:

“C(═O)(C(═O)C)O

C(═O)(C(O)C(═O)C)O

C(═O)(C(O)C(═O)C)O

C(═O)(C(O)C)O

. . . ”

Central to the working of the program is a computer simulation of the chemical reactions (i.e. transformations) which actually may take place during the Maillard process. Each virtual reaction or transformation is coded as a programme function that conducts the following steps:

1. 2-D pattern match on substrate (input) molecule(s) according to the virtual reaction

2. Break bonds

3. Change atom hybridisation

4. Change bond types

5. Add bonds

6. Output product molecule(s)

In principle, this may be coded in any suitable computer-readable format, for example in SPL (Sybyl Programming Language^{[3]}) or any equivalent way. Such a programme may require a coding of the molecules and transformations or computer operations, which can be done e.g. in SMILES^{[8]} or SLN (the line notation from Tripos^{[3]} which is better compatible with SPL), which are then applied in the code for the Reaction Set.

The pattern matching step allows for fragment matching on the connection table of the reactive fragment necessary for the reaction to take place. Thus the Maillard process is coded as a set of generic reactions which can act on a range of different starting molecules.

The IRG iterates through the Reaction Set, selecting reactions from the list of reactions and molecules from the ‘Soup’ that relate to that reaction. Preferably, a mass limit on the molecules in the Soup prevents polymerisation and focuses on volatile production.

The theory for kinetics for a simple chemical reaction: A+B→P, where A and B are substrates and P is the product molecule is:

$$\frac{ⅆ [ P ]}{ⅆ t} = \frac{- ⅆ [ A ]}{ⅆ t} = {k}_{ABP} \cdot [ A ] \cdot [ B ]$$

where k_{ABP} is the rate constant for that reaction. It is in principle possible, but very time consuming, to calculate the rates of chemical reactions in solution or in an enzymatic environment from the free energy profile along the reaction coordinate. The free energy of activation has a simple relation to the rate constant in the transition state approximation:

$${k}_{ABP} = \frac{{k}_{B} \cdot T}{h} {ⅇ}^{\frac{Δ G \#}{RT}}$$

Where

k_{B}=Boltzmann constant

T=temperature

H=Planck's constant

ΔG^{#}=free energy of activation

R=gas constant

ΔG^{#} consists of two components, the intrinsic part and the difference in free energy of salvation between the transition state and the reactants. The first can be calculated by either ab-initio or semi-empirical molecular orbital methods for both the transition state and the reactants. The difference in the free energies of solvation can be estimated using discrete solvent molecules or by continuum models. However the main obstacle, even with the fastest computers, remains the search for the transition state. Given the fact that we are dealing with more than hundred individual reaction steps this is a huge task. Therefore, in the present invention, it was decided that simulation becomes the preferred option. As a result a ‘reaction probability’ route approach has been adopted, using best guesses initially and preferably refining these empirically and/or by optimisation methods.

Discretising equation (1) the following is obtained:

Δ[A]=−k_{ABP}.[A].[B].Δt

Losing the time step Δt in the constant of proportionality, and describing values as probabilities, this may be written as:

Δ(n(A))∝−p(R_{ABP}).p(A).p(B)

where

n(A)=number of molecules of A in the Soup

p(R_{ABP})=relative ‘probability’ of Reaction A+B→P

p(X)=probability of selecting molecule X from the Soup

The joint probability p(A).p(B) may be simulated by randomly picking a pair of molecules {<molecule1>, <molecule2>}. This selection is biased by the ‘concentrations’ of molecule1 and molecule2 in the soup and therefore, over successive selections, is a reasonable approximation to the probability. p(R_{ABP}) may be simulated by assigning a ‘probability of reacting’ to each reaction R, and randomly selecting the reactions. If the selected molecules match the requirements of the reaction R then they react and the products are added to the soup. In essence this is simulating that if A & B come into contact in the ‘soup’: if they can react they should do so biased by some likelihood.

To facilitate scale-up, reduce computation time, and driving the simulation towards volatile products (which are the ones of interest in the present case), the reaction database (which is part of the reaction set) is preferably split into blocks, so that only selected reactions will occur within each block. The output from each block of reactions serves as input to one or more further blocks.

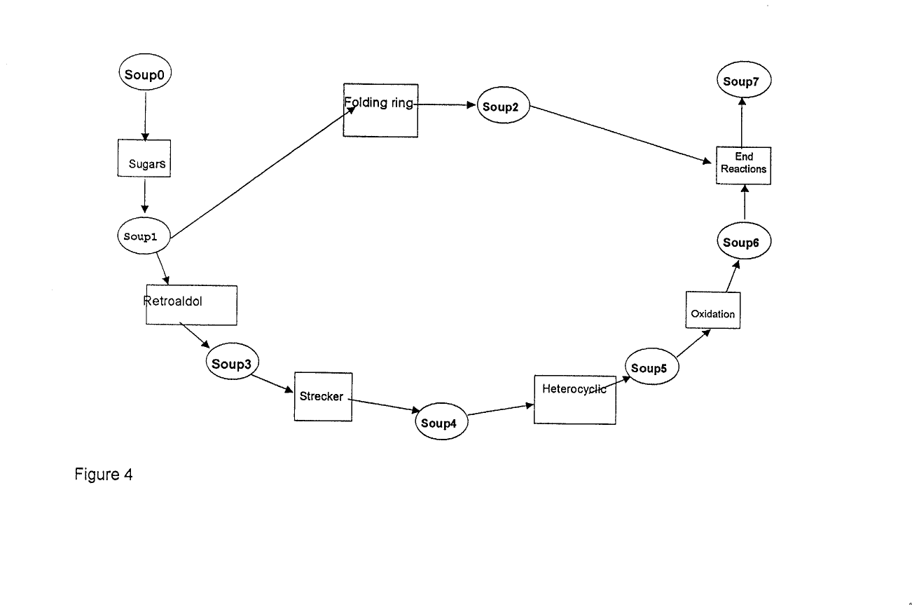

This is structured in FIG. 4 according to the order in which reactions occur in the Maillard process. This refinement is not as strongly sequential as it may appear: parallel reactions may take place within each block; the same reaction may occur in more than one block; and there is a high level of traffic between the blocks.

Alternatively to simulation of the reactions, estimations for determining one or more of the N processing parameters for obtaining the desired volatile component in a process for preparing a volatile component as set out herein before are derivable from a relationship between sensorical data, composition analyses of volatile compounds, and processing parameters used for obtaining the volatile compounds, said composition analyses being an actual mass distribution obtainable from performing at least 100 (preferably at least 1000) reactions involving heating reactants as herein defined under predetermined and known processing parameters, analysing the volatile fraction of the reaction product obtained form each of the reactions above to provide composition analyses thereof, encoding it as a mass distribution. Such mass distribution is obtainable from experimental results. In order to achieve this, samples may be produced under well defined standard conditions. The actual mass distribution may be obtainable by conventional chemical analysis of the reaction products or the volatile fraction thereof, such as GC and/or MS techniques. If so desired, this may be combined by computerised processing of the analytical data. Needless to say, in view of the large number of experiments to be carried out, this (conducting the experiments and analysis) is preferably carried out in a robotised or automated way.

In a preferred embodiment, in brief, a mixture of amino acid(s) and sugar(s) may be heated in solvent, cooled, and then extracted. The composition of volatile products may be determined by Gas Chromatography or similar separation technique. The identity of each peak may be determined by Mass Spectrometry from comparison with the generated fragmentation pattern of a library. From this a Molecular Mass Distribution (MMD) pattern can be reconstructed, representing the frequency of masses of the product composition of each individual experiment. The final output of the computational IRG contains the ‘soup’ of molecules at the end of the run. This may be represented as a “Virtual Mass Distribution” (VMD) by taking relative frequencies binned by molecular weight. The experimental MMD may then be compared with the VMD.

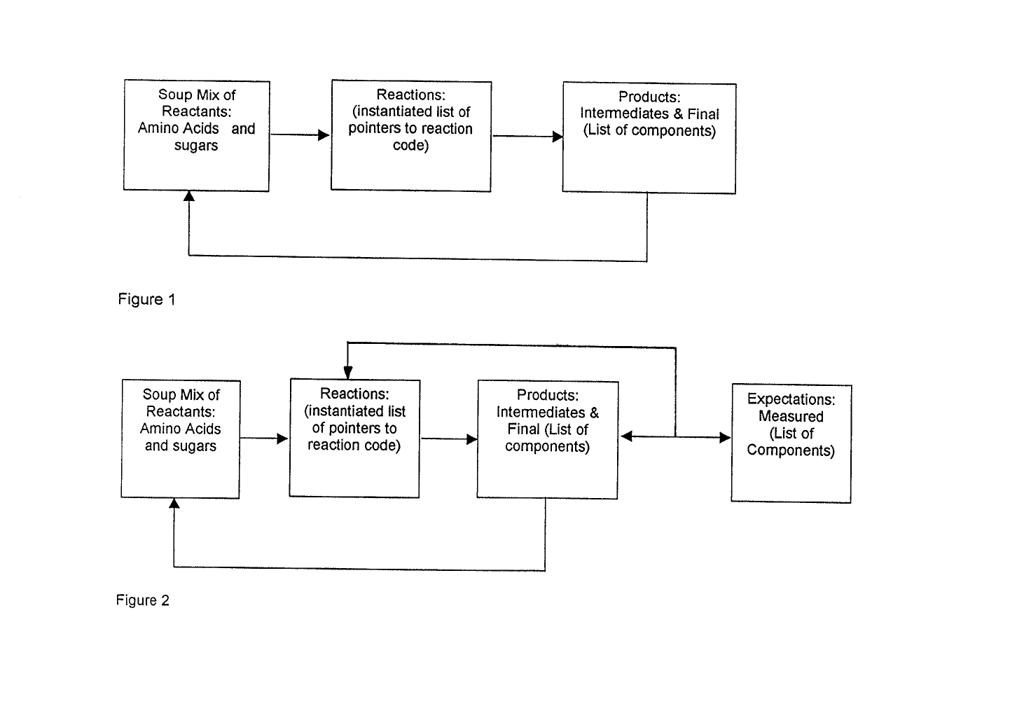

Comparison of the experimental (=actual) mass distribution with the virtual mass distribution, as generated using IRG, yields information that can be used to update the IRG and/or reaction set. E.g., compounds which show up in the experimental results but are missing in the IRG results might implicate that an elementary transformation is missing in the reaction database. Compounds present in the IRG results which are missing in the experimental mass distribution may originate from a probability of a certain transformation which is too high. The information thus acquired combined with the chemical knowledge of the user can be used to add or remove transformation steps and/or to change the probablities of some of the transformations, as is schematically given in FIG. 2.

The results described above, along with the full listing of the reactions paths, may be used as a guide to identifying where the output of the IRG may be improved by updating the values of the reaction rate parameters. The effect of such updates may easily be evaluated by running the updated IRG and comparing the results with the experimental data. If this results in an improvement the update is accepted, otherwise other updates are attempted.

The invention further relates to a computerized system comprising means for entering sensorical data, GC (‘fingerprint’) data and process variables to be set at the start of a chain of reactions, and a computer programme for predicting process variables to obtain new desired fingerprint data and/or sensorical data using an iterative procedure, based upon already entered sensorical data, fingerprint data and process variables, and means for providing output.

In a preferred embodiment, the comparison or relationship between sensorical data, composition analyses of volatile compounds in the form of actual and/or virtual mass distributions, and processing parameters used for obtaining the composition analysis are obtainable using statistical methods. An example of such statistical methods may be a relationship method like linear- or non-linear regression, PLS, neural networks, gaussian procedures, etcetera.

The reaction rate parameters (probabilities) may be optimised by any suitable method. For example, the method as described below may be used.

First an objective or cost function related to the experimental measures is defined as:

Error(R(pH, T), S)=false_positives(S, pH, T)+false_negatives(S, pH, T);

where

R=the set of transformation rate parameters (i.e. probabilities) at the specified pH [high, med or low] and T (temperature of soup)

S=the start soup

false_positives=the number of molecules the IRG has incorrectly identified as being present in the final soup

false_negative=the number of molecules the IRG has failed to identify as being present in the final soup

Note that this does not take into account the peak height, but only the presence or absence of particular molecules. Then an objective function summed over the start soups for which there is experimental data may be defined:

O(R(pH, T))=Σₛ Error(R(pH, T), S)

Clearly as O(R(pH, T)) approaches 0, the IRG is producing results closer to the experimental values. Defining the optimisation problem to be to optimise R(pH, T), i.e. the rate parameters for a given pH and temperature, such that O(R(pH, T)) is minimised. This is computationally expensive but may be achieved using a standard optimisation algorithm such as Sequential Quadratic Programming or a Genetic Algorithm.

Comparing the virtual mass distribution with the actual molecular mass distribution may be further supplemented with analysis of and comparison with sensory data. Such sensory data may be obtained from analysing (e.g. using a sensory panel) the reaction products of the actual experiments, and preferably the volatile fraction thereof. The analysis of sensory data may involve statistical methods for mapping the sensory data. If sufficient data are then obtained, mathematical relationships between sensorical data and processing variables may then be derived.

### REFERENCES

[1] Molecular Simulations Inc., 9685 Scranton Road, San Diego, Calif. 92121-3752, USA.

[2] Oxford Molecular Group PLC, The Medaware Centre, Oxford Science Park, Oxford OX4, 4GA, United Kingdom.

[3] Tripos Inc., 1699 South Hanley Road, St. Louis, Mo. 63144, USA.

[4] Vernin, G.,; Parkyani C.; Barone R.; Chanon M.; Metzger J.; Computer Assited Organic-Synthesis of Volatile Hererocyclic Compounds in Food Flavours, journal of Agriculture and Food Chemistry, 1987, 35, 5 761-768.

[5] Azario, P.; Arbelot M.; Baldy A.,; Microcomputer Assisted Retrosynthesis (MARS). New Journal of Chemistry, 1990, 14, 12, 951-956.

[6] Bador, P. et al; Les Systtemes Informatiques de Recherche d'Information sur les Reacctions Chimiques et les Systems de Synethese Assistee par Ordinateur, New Journal of Chemistry, 1992, 16, 3, 413-423.

[7] Prickett, S. E.; Mavrovouniotis, M. L.; Construction of Complex Reaction Systems—II Molecule Manipulation and reaction applicatoin algorithms, Computers Chem. Engng., 1997, 21, 11, pp 1237-1254

[8] Weininger, D., SMILES, a chemical language and information syste, Journal of Cehmical Information and Computer Science, 1998, 28, 1, 31-36

[9] Lohn, J. D.; Evolving Catalytic Reaction Sets using Genetic Algorithms, IEEE World Congress on Computational Intelligence, Anchorage, Ak. 1998, 87-492

[10] Schuster, P.; Dynamical Systems an dCellular Automata, J. Demongeot et al. (Eds), Academic Press, 1985, 255-267

[11] banzhaf, W. et al; Emergent Computation by Catalytic Reactions, Nanogechnology, 1996, 7, 307-314

[12-Kauffman, S. A.; The Origins of Order, Oxford University Press. 1993, 303-305

[13] H.-D. Belitz and W. Grosch, Food Chemistry, Chapter 4, Carbohydrates 200-256, Verlag. (translatoin from the second German Edition by D. Hadziyev).

[14] F. Ledl and E. Schleicher, Angew. Chem. Int. Ed. 29 565-594 (1990).

[15] V. A. Yaylayan and A. Hyuyghues-Despointes, Crit. Revs in Food Sci. and Nutr. 34, 321-369 (1994).

[16] J. Kerler, J. G. M. van der Ven, H. Weenen, Food Rev. Int., 13, 553-557 (1997).

[17] R. Tressl, B. Helak, E. Kersten and C. Nittka, Recent Developments in Flavour and Fragrance Chemistry, Proceeedings of the 3^{rd} International Symposium, 167-181. Ed. R. Hopp and K. Mori, VCH Verslagsgeselschaft mbH, 1993.

[18] R. Tressl and D. Rewicki, Flavor Chemistry: 30 Years of progess, 305-325 edited by Teranishi et al. Kluwer Academic/Plenum Publishers, New York 1999.

### EXAMPLES

### Example 1

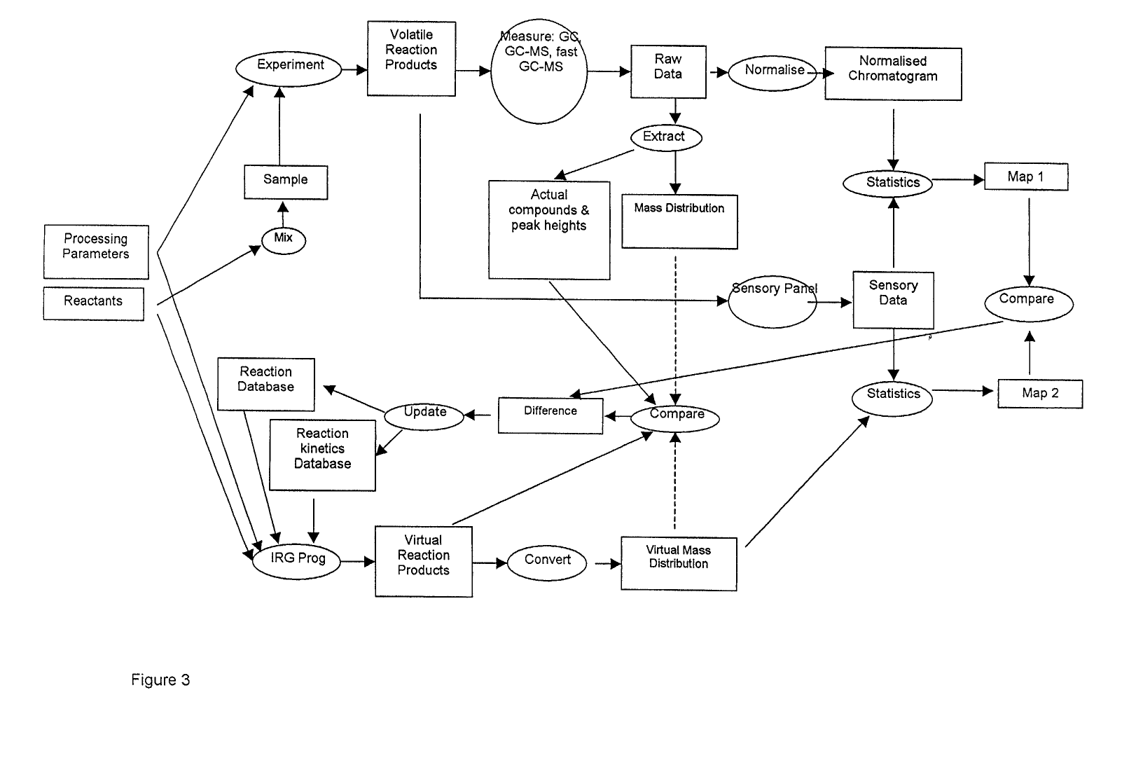

In FIG. 3m, an example is given how an assembly of actual and virtual experimentation, and sensory analysis may be used jointly.

### Example 2

This exmaple gives a high level pseudocode for how the IRG may be coded.

Initialise Soup, Reaction Set

Loop
Loop through Reaction Blocks*

Select Random reaction
If (transformation probability > random number)

Select random reactant(s)
If reactant(s) are correct for reaction

Remove bonds
Change atom type & hybridisation
Add bonds
If (mass of product < mass limit)**

Remove reactants from Soup
Add product(s) to Soup

Endif

Endif

Endif

Endloop
Endloop
Italics indicate optional computer instructions:
*if reaction blocks are used
**if a mass limit is used

### Example 3

This example gives the SPL code for the main body of the IRG, similar to Example 2

```
#============================================================#
uims define expression_generator iterate yes
  setvar fh %open($filename3)
  setvar fh2 %open($filename5)
  %write($fh2 Time $chkprod)
# Call blocks of reactions.
FOR blocks in %range(1 $blocknum 1)
  %write($fh “ ”)
  %write($fh “Block” $blocks)
  %write($fh “ ”)
  %write($fh2 “ ”)
  %write($fh2 “Block” $blocks)
  %write($fh2 “ ”)
  setvar inns %set_unpack($inputset[$blocks])
  FOR those in $inns
  setvar soupmix[$blocks] $soupmix[$blocks] $soupmix[$those]
  ENDFOR
#iterate on soupmix[$blocks]
    FOR backups in %range(1 10 1)
```

```
FOR u in %range(1 10 1)
  setvar v 0
  FOR t in %range(1 %math($icycles / 100) 1)
```

setvar randomnu %math($lastprob[$blocks] * %rand())
setvar reactionnumber “”
FOR roulette in %range($totalnum[$blocks] 1 −1)

IF %LTEQ($randomnu $cumulist[$blocks][$roulette])

setvar reactionnumber $roulette

ENDIF

ENDFOR

setvar runreaction %arg($reactionnumber $totallist[$blocks])
setvar reacttype %substr($runreaction 1 2)
IF %streql(R1 $reacttype)

# Call unimolecular reaction with random reactants

FOR alpha in %range(1 4 1)

setvar soupsize %count($soupmix[$blocks])
setvar j %math(%int(%math(%math($soupsize − 0.0002) * %rand())) +

1.0001)

setvar soupmol %arg($j $soupmix[$blocks])

IF %gt(%strlen($soupmol) 0)

```
  setvar scommand %cat(‘%’ $runreaction ‘(‘“ $soupmol ”’)’)
setvar mproduct %eval($scommand)
  IF %gt(%strlen($mproduct) 1)
```

setvar soupmix[$blocks] %item_remove($j $soupmix[$blocks])
setvar mproduct %remwater(“$mproduct”)
setvar soupmix[$blocks] $soupmix[$blocks] $mproduct

```
  %uppaths($soupmol $runreaction “$mproduct”)
  %uptable($soupmol $runreaction “$mproduct”)
  %upretable($runreaction)
  setvar v %math($v + 1)
```

ELSE
ENDIF

ENDIF

ENDFOR

ELSE

#Call bimolecular reaction with random selections of two reactants

IF %streql(R2 $reacttype)

FOR alpha in %range(1 4 1)

setvar soupsize %count($soupmix[$blocks])
setvar n %math(%int(%math(%math($soupsize − 0.0002) * %rand())) +

1.0001)

setvar first %arg($n $soupmix[$blocks])
setvar j %math(%int(%math(%math($soupsize − 0.0002) * %rand())) +

1.0001)

IF %eq($j $n)
ELSE

setvar second %arg ($j $soupmix[$blocks])
IF %gt(%strlen($first) 0)
IF %gt(%strlen($second) 0)

setvar soupmols %cat($first . $second)
setvar scommand %cat(‘%’ $runreaction ‘(‘“ $soupmols ’”)’)
setvar mproduct %eval($scommand)
IF %gt(%strlen($mproduct) 1)

IF %gt($n $j)

setvar soupmix[$blocks] %item_remove($n

$soupmix[$blocks])

setvar soupmix[$blocks] %item_remove($j

$soupmix[$blocks])

ELSE

setvar soupmix[$blocks] %item_remove($j

$soupmix[$blocks])

setvar soupmix[$blocks] %item_remove($n

$soupmix[$blocks])

ENDIF
setvar mproduct %remwater(“$mproduct”)
setvar soupmix[$blocks] $soupmix[$blocks] $mproduct
%uppaths($first $runreaction “$mproduct”)
%uptable($first $runreaction “$mproduct”)
%uppaths($second $runreaction “$mproduct”)
%uptable($second $runreaction “$mproduct”)
%upretable($runreaction)
setvar v %math($v + 1)

```
  ELSE
  ENDIF
ENDIF
```

ENDIF
ENDIF

ENDFOR
ENDIF

ENDIF
ENDFOR
setvar chksum “”

# check for the presence of compounds in current soupmix.

IF %streql(yes $pcheck)

FOR x in %range(1 %count($soupmix[$blocks]))

setvar dummy %smiles_to_mol(m1 %arg($x $soupmix[$blocks]))
FOR y in %range(1 %count($chkprod))

IF %sln_search2d(m1 %arg($y $chkprod) mutual norm 1)

IF $chksum[$y]

setvar chksum[$y] %math(1 + $chksum[$y])

ELSE

setvar chksum[$y] 1

ENDIF

ENDIF

ENDFOR

ENDFOR

ENDIF
%write($fh2 %arg(4 %time()) $chksum)
%write($fh %arg(4 %time()) $v)

ENDFOR

# Make a temporary save of the soupmix and paths

```
  echo “Saving backup file ...”
  %tmp_file_save(%math($backups * 10) $blocks $backupname)
echo “Backup file saved.”
```

ENDFOR

IF %streql(yes $timevms)
# Write multiple virtual mass spec graph data to file
# Uses the current block of the soupmix not rather than the whole.

```
  setvar size 1
  setvar mass “”
  setvar w %printf(“%02d” $blocks)
  setvar fh3  %open(%cat($vmsname $w .txt))
FOR j in %range(%count($soupmix[$blocks]) 1 −1)
```

setvar dummy %smiles_to_mol(m1 %arg($j $soupmix[$blocks]))
setvar mass[$j] %int(%molmass(m1))

ENDFOR
setvar mass %sortn($mass)
setvar n 1
FOR k in %range(%math(%count($mass) − 1)1 −1)

IF %eq(%arg($k $mass) %arg(%math($k + 1) $mass))

setvar n %math($n + 1)
setvar $mass %item_remove(%math($k + 1) $mass)

ELSE

%write($fh3 %arg(%math($k + 1) $mass) %math($n * $size))
setvar n 1

ENDIF

ENDFOR
%write($fh3 %arg(1 $mass) %math($n * $size))
%close($fh3)

ENDIF

ENDFOR
%close($fh2)
%close($fh)

### Example 4

Basic rules for writing each reactoin in SMILES notation and three exmaples of reactions typical for Maillard, as found in literature and how they are coded into SMILES strings and reactions for the IRG.

Basic rules for SMILES:

Instructions for adding to data base:

Is this an UNARY or a BINARY reaction type?

UNARY

R1_{—}1_{—}1_sugar

Pattern for matching against, atoms start counting at 0 from the left

Binary reactions have two patterns, atom numbers continue from the first pattern onto the second

C(═O)C(O)C(O)

The numbers of atoms which have restrictions to the atoms joined to them

−1 terminates the list

0 3 4 5 −1

These are the restrictions as atom type letter and hybridisation number

H3 H3 H3C3 H3

Other restriction state if at least one Hydrogen must be present

N N Y N

Catstring is for adding water if required, the number assigned to it follows on from the last atom of the pattern

Both unary and binary reactoin suse this. If not used then NA replaces it.

NA

bonds to be removed as the numbers of the atoms which are on each end

2

2 3

4 5

bonds to be added as the numbers of the atoms on each end with bondtypes

1

2 3 2

Note: The numbering in each of the 2D representations is the same as that used for the atoms on converting into SMILES notation.

### Example 4a

### R2_{—}3_{—}15_{—}1_pyrroline

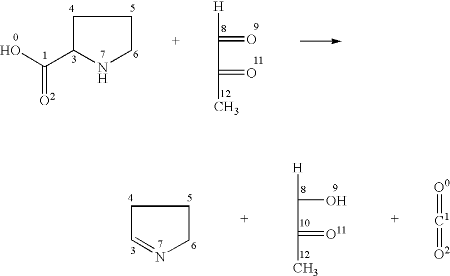

reaction in SMILES code:

BINARY

R2_{—}3_{—}15_{—}1_pyrroline

OC(═O)C1CCCN1

C(═O)C(═O)C

0 3 4 5 6 7 8 12 −1

H3 H3 H3 H3 H3 H3 H3 H3

N N N N N N N N

NA

4

0 1 1 3 3 7 8 9

3

0 1 2

3 7 2

8 9 1

Added Apr. 27, 1999 (SR)

J. E. Hodge, F. D. Mills and B. E. Fisher, Cereal Sci. Today 17, 34-40 (1972)

Checked Oct. 5, 1999 (FH)

### Example 4b

### R2_{—}10_{—}1r_rS+AAMeCHOpyrrol

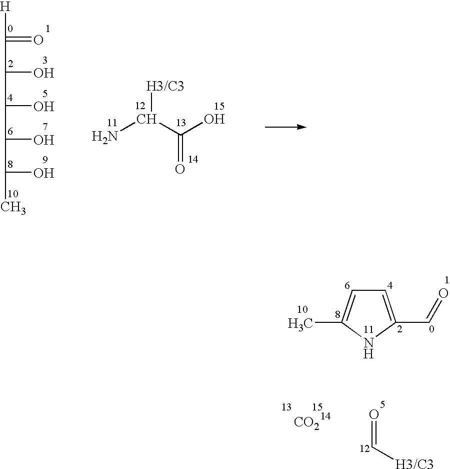

BINARY

R2_{—}10_{—}1b_rS+AAMeCHOpyrrol

C═O)C(O)C(O)C(O)C(O)C

NCC(═O)O

0 3 5 7 9 10 11 12 15 −1

H3 H3 H3 H3 H3 H3 H3 H3C3 H3

N N N N N N N Y N

NA

9

2 3 2 4 4 5 6 7 6 8 8 9 11 12 12 13 13 15

6

2 4 2

2 11 1

6 8 2

8 11 1

12 5 2

13 15 2

water molecules not explicitly drawn

Added Sep. 9, 1999 (SR). Comparable to R2_{—}10_{—}1b_asugarAA but on rhamnose.

R. Tressl, E. Kersten, C. Nittka and D. Revicki. Maillard Reactions in Food and Health, Proceedings of 5th Int. Symp. on Maillard Reactions Aug. 26-Sep. 1, 1993. (RSC Special publication 151, 1994, p. 51)

### Example 4c

### R2_{—}8_{—}14b_{—}2thiopent3on

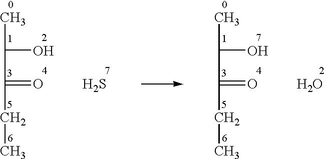

BINARY

R2_{—}8_{—}14b_2thiopent3on

CC(O)C(═O)CC

S

0 1 2 5 6 7 −1

H3 H3 H3 H3 H3 H3

N N N N N N

NA

1

1 2

1

1 7 1

Added Aug. 8, 1999 (FH)

changed to OH/SH-substitution J. Agric. Food Chem. 1999, 47, 1626.—Aug. 25, 1999 (FH)

### Example 5

Exmaple of blocks of reactoins as may be used in the reactoin database, according to the order in which reactions occur in the Maillard process, but the same reaction may occur in more than one block (FIG. 4). Other arrangements are possible.

### Example 6

Experimental validation with virtual mass distribution (VMD) was obtained by comparison of an actual mass distribution (MMD) with a virtual mass distribution. The conditions for the simulatoins were: 100 molecules glucose, 100 molecules threonine, 6000 iterations, pH=7, Temperature=120° Celsius. The conditions for the real experiment are: equimolar mixture of glucose and threonine, in a buffered solution pH=7, processed during 1 hour at 120° Celsius.

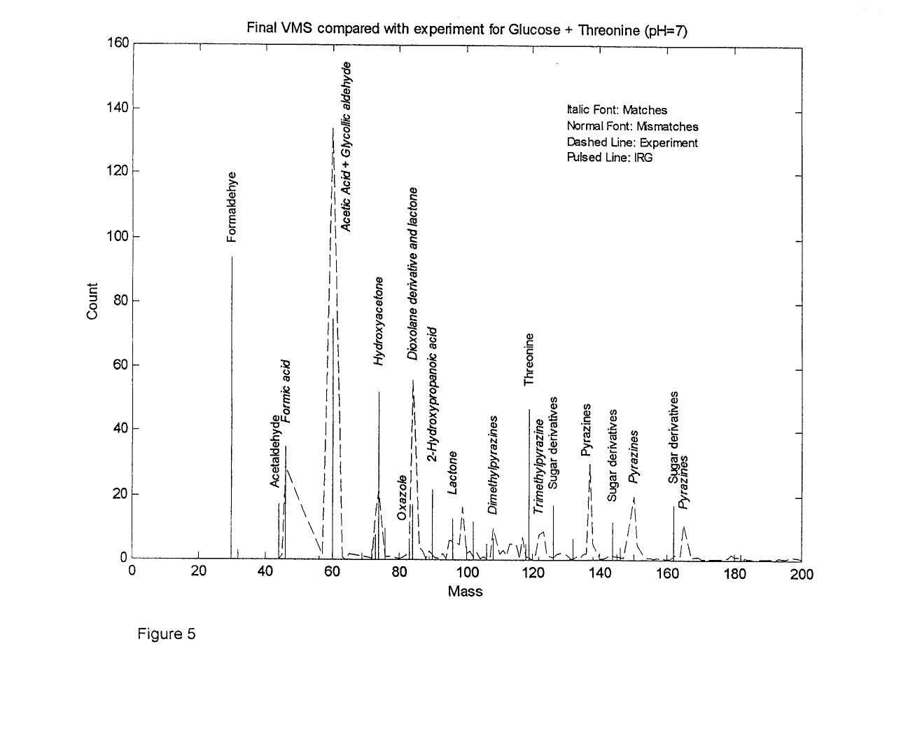

In FIG. 5, the MMD, the VMD, and the matches have been printed in different fonts. Clearly, the formation of formic acid, acetic acid, glycolic aldehyde, hydroxyacetone, lactons, oxazoles, and some pryazines can be seen. There are also a number of mismatches: a number of start components and intermediates, such as threonine, formaldehyde, acetaldehyde, and various sugar derivatives are present in the IRG ‘soup’ but not in the experimental results. The IRG has also fialed to match some the substituted pryazines as well as some of the smaller peaks.

1. Process for preparing a volatile component or mixture of components, said process comprising heating a mixture containing reactants as herein defined, said heating being performed under N different processing parameters wherein one or more of the N processing parameters and/or one or more of the reactants to choose for obtaining the desired volatile component are derivable from a relationship between:
sensorical data obtainable from analysing reaction products of actual experiments,
volatile reaction products and their composition,
processing parameters,
reactants,
said composition being obtainable:
(a) from an actual mass distribution obtainable from performing at least 100 (preferably at least 1000) reactions involving heating reactants under predetermined and known processing parameters, analysing the volatile fraction of the reaction product obtained from each of the reactions above to provide composition analyses thereof, encoding it as a mass distribution, and/or
(b) simulation of complex chemical reaction pathways involving the simulation of reacting reactants under predetermined and specified processing parameters.

2. Process according to claim 1, wherein said N different processing variables may comprise at least one of concentration of reactants, heating temperature, heating time, pH of the reaction mixture, water activity.

3. Process according to claim 1, wherein the reactants comprise minimally a sugar or carbohydrate source together with a nucleophilic species, such as amino acid(s), or sources of amino acids such as peptides or proteins and optionally further containing salts, cosolvents, buffers, aldehydes, ketones, alcohols, amines, acids, esters, lactones, cyclic heterocycles, thiols, ethers, thioethers or mixtures thereof.

4. Process according to claim 1, wherein the actual mass distribution of step (a) are obtainable by conventional chemical analysis of the reaction products or the volatile fraction thereof.

5. Process according to claim 4, wherein the conventional chemical analysis of step (a) involves Gas Chromatography and/or Mass Spectroscopy techniques.

6. Process according to claim 4, wherein the chemical analysis of step (a) is combined by computerised processing of the analytical data.

7. Process according to claim 1, wherein the reactions performed in step (a) to obtain the actual mass distribution are carried out in a robotised way.

8. Process according to claim 1, wherein the simulation of complex chemical reaction pathways in step (b) is obtainable by iteratively applying a set of operations or computer intructions using a computer programme to:
A ‘Soup’ of molecules representing the current state of the system
A ‘Reaction Set’ describing transformations and corresponding probabilities that may take place in the Maillard process
to produce volatile and non-volatile molecules, for simulating complex chemical reactions when such product is run on a computer, and wherein the iteration is effected by a computer programme directly loadable in the internal memory of a computer, and wherein the computer programme contains two main elements:
computer instructions for running the reactions using the Reaction Set,
computer instructions for the iterative procedure of selecting molecules, running the reactions and producing output.

9. Process according to claim 8, wherein during the iterative procedure the newly formed compounds are added back to the Soup, and the volatile components which are present during the simulation form (part of) the virtual mass distribution.

10. Process according to claim 8, wherein the Soup at the start of the reaction is equal to the starting mixture of molecules.

11. Process according to claim 1, wherein iterative operation in step (b) is a computer-readable format encoded by:
Loop
Loop through reaction blocks
Select Random reaction
if (transformation probability > random number)
Select random reactant(s)
If reactant(s) are correct for reaction
Remove bonds
Change atom type & hybridisation
Add bonds
If (mass of product < mass limit)
Remove reactants from Soup
Add product(s) to Soup
Endif
Endif
Endif
Endloop
Endloop
or any functional equivalent thereof, wherein the Italics indicate optional computer instructions.

12. Process according to claim 1, wherein both the actual and the virtual mass distributions are obtained, and wherein the actual mass distribution is compared with the virtual mass distribution, and wherein the generated actual mass distribution is used to update the IRG and/or Reaction Set.

13. Process according to claim 1, wherein the relationship between one or more of:
sensorical data,
volatile reaction products and their composition,
processing parameters,
reactants
are obtainable using statistical methods.

14. Process according to claim 13, wherein the statistical method is one of linear- or non-linear regression, PLS, neural networks, gaussian procedures.

15. A computer program product directly loadable into the internal memory of a digital computer, comprising software code portions for the simulation of complex chemical reaction pathways by iteratively applying a set of operations or computer intructions to:
a Soup of molecules representing the current state of the system,
a Reaction Set describing transformations that may take place in the Maillard process,
to produce volatile and non-volatile molecules, wherein the iteration is coded as a computer programme, for simulating complex chemical reactions when such product is run on a computer.

16. Computerized system comprising means for entering sensorical data, fingerprint data and process variables to be set at the start of a chain of reactions, and a computer programme for predicting process variables to obtain new desired fingerprint data and/or sensorical data using an iterative procedure, based upon already entered sensorical data, fingerprint data and process variables, and means for providing output.

17. A computer programme product directly loadable into the internal memory of a digital computer, comprising software code portions coding for:
Initialise Soup, and Reaction Set (containing reaction database and reaction kinetic database)
Loop
Loop through reaction blocks
Select Random reaction
If (transformation probability > random number)
Select random reactant(s)
If reactant(s) are correct for reaction
Remove bonds
Change atom type & hybridisation
Add bonds
If (mass of product < mass limit)
Remove reactants from Soup
Add product(s) to Soup
Endif
Endif
Endif
Endloop
Endloop
when such product is run on a computer, wherein the Italics indicate optional computer instructions.
